(19) United States
(12) Patent Application Publication
(10) Pub. No.: US 2024/0122937 A1
(43) Pub. Date: Apr. 18, 2024

(54) TREATING CANCER IN PATIENT WITH PTEN INACTIVATING MUTATION

(71) Applicant: TAIHO PHARMACEUTICAL CO., LTD., Chiyoda-ku (JP)

(72) Inventors: Karim BENHADJI, Princeton, NJ (US); Osamu TAKAHASHI, Princeton, NJ (US)

(73) Assignee: TAIHO PHARMACEUTICAL CO., LTD., Chiyoda-ku (JP)

(21) Appl. No.: 18/263,613

(22) PCT Filed: Jan. 27, 2022
(86) PCT No.: PCT/US2022/014125

§ 371 Date: Jul. 31, 2023

Related U.S. Application Data
(60) Provisional application No. 63/144,153, filed on Feb. 1, 2021.

(51) Int. Cl. A61K 31/5365; A61P 35/00

(52) U.S. Cl. CPC A61K 31/5365; A61P 35/00

(57) ABSTRACT

A method of treating a subject with a cancerous tumor exhibiting an inactivating mutation in the PTEN gene including administering to the subject trans-3-amino-1-methyl-3-(4-(3-phenyl-5H-imidazo[1,2-c]pyrido[3,4-e][1,3]oxazin-2-yl)phenylcyclobutanol or a pharmaceutically acceptable salt thereof.

### BACKGROUND OF THE INVENTION

### Field of the Invention

The present invention relates to treating a cancerous tumor harboring an inactivating mutation in the PTEN gene.

### Description of the Related Art

PTEN (Phosphatase and tensin homolog) encodes a ubiquitously expressed phosphatase that counteracts the PI3K/AKT/mTOR cascade—one of the critical growth-promoting signaling pathways. A PTEN inactivating mutation can cause a spectrum of phenotypes including benign overgrowths, malignancies, and metabolic and neurodevelopmental disorders. PTEN dysfunction can be caused by a germline mutation (hereditary) and a somatic mutation (acquired). The somatic PTEN mutation may impact the tumor tissues where the mutation occurred, while the germline PTEN mutation is not restricted as to the tumor tissues that may be impacted.

A frequent PTEN-related hereditary condition is Cowden Syndrome (CS), an autosomal dominant multi-system disorder characterized by multiple hamartomas and increased lifetime risks of breast, thyroid, and other cancers. Other clinical phenotypes involve multiple organ systems such as the brain, uterus, colon, and mucocutaneous tissues. CS is an extremely rare disease, and the prevalence is estimated at 1 in 200,000 individuals.

PTEN inactivating mutations are associated with elevated lifetime risks of breast cancer (estimated lifetime risk of 85%), thyroid cancer (35%), renal cell carcinoma (34%), endometrial cancer (28%), colorectal cancer (9%), and melanoma (6%). Moreover, individuals with germline PTEN mutations have a 7-fold increased risk of developing a second primary malignant neoplasm compared to the general population in the United States.

Treating cancerous tumors in patients with inactivating PTEN mutations is discussed in Xing et al. Breast Cancer Research (2019) 21:78, and Kingston et al. JCO Precision Oncol. 2019; 3:1-7. Other PTEN-related heredity conditions include Bannayan-Riley-Ruvalcaba syndrome (BRRS), PTEN-related Proteus syndrome (PS), Proteus-like syndrome (PS-like).

Applicant has conducted research and investigation and provides a method of effectively treating cancers in individuals with germline PTEN inactivating mutations.

Applicant has conducted research and investigation and provides a method of effectively treating cancers in individuals with somatic PTEN inactivating mutations.

### SUMMARY OF THE INVENTION

An aspect of the present invention is to provide a method for treating a subject with a cancerous tumor which exhibits a germline or somatic inactivating mutation in the PTEN gene by administering to the subject the compound trans-3-amino-1-methyl-3-(4-(3-phenyl-5H-imidazo[1,2-c]pyrido[3,4-e][1,3]oxazin-2-yl)phenylcyclobutanol or a pharmaceutically acceptable salt thereof. This compound having the formula (1) below is referred to as Compound (1):

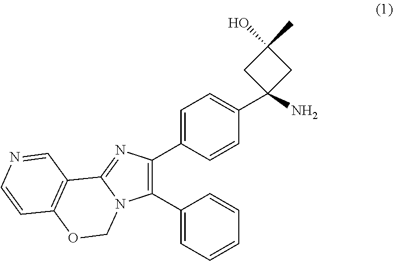 [SMILES: C[C@]1(O)C[C@@](N)(c2ccc(-c3nc4n(c3-c3ccccc3)COc3ccncc3-4)cc2)C1]

The cancers that can be treated include breast cancer, thyroid cancer, renal cell carcinoma, endometrial cancer, colorectal cancer, and melanoma, and glioblastoma (brain tumor) plus other cancers having an inactivating mutation of the PTEN gene.

An embodiment of the present disclosure provides the following items.

Item 1. A method of treating a subject with a cancerous tumor, comprising: determining if the cancerous tumor exhibits an inactivating PTEN mutation; and administering to the subject with the cancerous tumor exhibiting the inactivating PTEN mutation Compound (1) or a pharmaceutically acceptable salt thereof.

Item 2. The method of item 1 wherein the inactivating PTEN mutation is somatic.

Item 3. The method of item 1 wherein the administering is daily for at least 21 days.

Item 4. The method of item 1 wherein the administering is for 4 consecutive days followed by a 3-day drug holiday in a week.

Item 5. The method of claim 4 wherein the administering is repeated for a total period of at least 21 days.

Item 6. A method of treating a subject with a cancerous tumor, comprising:
determining if the subject has a PTEN gene with a germline inactivating mutation; and
administering to the subject having the PTEN gene with the germline inactivating mutation Compound (1) or a pharmaceutically acceptable salt thereof.

Item 7. The method of item 6 wherein the administering is daily for at least 21 days.

Item 8. The method of item 6 wherein the administering is for 4 consecutive days followed by a 3-day drug holiday.

Item 9. The method of item 8 wherein the administering for 4 consecutive days followed by the 3-day drug holiday is repeated for a total period of at least 21 days.

Item 10. An anti-cancerous tumor agent comprising Compound (1) or a pharmaceutically acceptable salt thereof for treating a subject with a cancerous tumor which exhibits an inactivating PTEN mutation.

Item 11. Compound (1) or a pharmaceutically acceptable salt thereof for use in treating a subject with a cancerous tumor which exhibits an inactivating PTEN mutation.

Item 12. Use of Compound (1) or its pharmaceutically acceptable salt for producing an anti-cancerous tumor agent used administering to a subject with a cancerous tumor which exhibits an inactivating PTEN mutation.

### DETAILED DESCRIPTION OF THE PREFERRED EMBODIMENTS

Compound (1) is described in U.S. Pat. Nos. 8,772,283, 9,375,434, 10,155,990 and 10,323,044 the contents of which are incorporated herein by reference in their entirety.

In the present application, the term “administration schedule” is a plan in which the type, amount, period, procedure, etc. of the drug in the drug treatment are shown in time series, and the dosage, administration method, administration order, administration date, and the like of each drug are indicated. The date specified to be administered is determined before the start of the drug administration. The administration is continued by repeating the course with the set of administration schedules as “courses”. The administration schedule in which the drug administration and the specified rest are repeated as one course is shown as 2 courses (administration schedule for repeating the course of one course twice), three courses (administration schedules repeated three times), and the like.

In the present application, “administration” indicates that the drug is administered, and the term “rest” indicates that no drug is administered. The term “administration day” is the day to be administered, and is the day that is not administered to the “rest date” The term “defined rest” or “defined rest period” used in the present application indicates that a drug is not administered in a predetermined administration schedule or indicates the period.

The cancers treated are those where the PTEN gene has one or more inactivating mutations. The cancers which can be treated include head and neck cancer (e.g., oral cancer, pharyngeal cancer, laryngeal cancer, nasal cancer, sinus cancer, salivary gland cancer, thyroid cancer), gastrointestinal cancer (e.g., esophageal cancer, stomach cancer, duodenal cancer, liver cancer, biliary tract cancer (e.g., gall bladder/bile duct cancer), pancreatic cancer, colorectal cancer (e.g., colon cancer, rectal cancer)), lung cancer (e.g., non-small cell lung cancer, small cell lung cancer, mesothelioma), breast cancer, renal cell carcinoma, genital cancer (e.g., ovarian cancer, uterine cancer (e.g., cervical cancer, endometrial cancer)), urinary cancer (e.g., kidney cancer, bladder cancer, prostate cancer, a testicular tumor), a hematopoietic organ tumor (e.g., leukemia, malignant lymphoma, multiple myeloma), a bone/soft tissue tumor, skin cancer (e.g., melanoma, non-melanoma skin cancer) and a brain tumor (e.g., glioblastoma,), preferably head and neck cancer, breast cancer, renal cell carcinoma, genital cancer, gastrointestinal cancer, skin cancer, a brain tumor, more preferably breast cancer, thyroid cancer, renal cell carcinoma, endometrial cancer, colorectal cancer, melanoma, and glioblastoma. The inactivating mutation can be a germline mutation or a somatic mutation. A cancer can be determined to have a somatic mutation by genotyping a sample from the tumor for a PTEN inactivating mutation. The PTEN alterations can be its loss at DNA, RNA or protein (loss of target) and/or loss of function caused by inactivating mutations (gene/protein is there but altered) A germline alterations can be determined by molecular profiling techniques at DNA, RNA or protein level as for somatic alterations or from the family history. The cancers treated are usually solid cancers.

The subject having a cancerous tumor with one or more inactivating mutations of the PTEN gene can be treated with Compound (1) or its pharmaceutically acceptable salt at dose levels of from about 1 mg/day, about 4 mg/day, about 16 mg, about 32 mg/day, about 44 mg/day, and 160 mg/day. The dosing level may be varied within the ranges such as from about 1 mg/day to about 160 mg/day, from about 4 mg/day to about 160 mg/day, from about 4 mg to about 44 mg/day, and from about 16 mg/day to about 32 mg/day. The dosing can be daily or intermittently. Doses of about 8 and 12 mg/day QD and 16 mg and 20 mg/day for intermittent dosing are preferred. The daily dose may be administered in divided doses. If intermittently, the schedule may be 4 days of administration and 3 days off (drug holiday) in a week. The dosing whether continuous or intermittent is continued for a 21 day cycle. The cycle may be repeated without a drug holiday or with a drug holiday depending upon the subject. Other schedules are possible depending upon the subject's response to the drug. The larger doses are usually given intermittently with doses up to about 24 mg usually given daily. A subject may be started with a low dose and then have the dose escalated until either maximum dose is reached or the subject experiences adverse events at which point the escalation is stopped and the drug dosing reduced to a previous dose where the adverse was not experienced or was not serious enough to require stoppage of the treatment. Typical dosing for the continuous regimen may be from 8 to 24 mg/day, preferably 8, 12, 16, 20 or 24 mg/day but higher or lower doses may be used depending on the subject's response to the treatment and presence or absence of adverse events. More preferably, the dosing for the continuous regimen is 8, 12, 16, or 20 mg/day, more preferably, 12, 16, or 20 mg/day, more preferably, 16 or 20 mg/day. The typical dosing for the intermittent schedule is 8 to 32 mg/day, preferably 16, 20, 24, 28, or 32 mg/day. More preferably, the dosing for the intermittent schedule is 16, 20, 24, or 28 mg/day, more preferably, 20, 24, or 28 mg/day, more preferably, 24 mg/day. The dosing may be adjusted as desired depending on the subject's response and presence or absence of adverse events. If a dose is well-tolerated the dose can be increased. The intermittent administration may be continued for 21 day which comprises one cycle, and the cycle may be repeated as desired.

As used in the present application, an “adverse event” refers to any unfavorable or unintended illness or symptom thereof occurring in a patient to whom a drug has been administered. It does not matter whether there is a causal relationship with the drug or not.

Compound (1) is not cleared from the body quickly but can accumulate in the body, allowing for intermittent administration. A 4 days on and 3 days off cycle in a week is one such cycle, the cycle may be modified to accommodate patients having pharmacokinetics allowing for more days on or requiring more days off such as 5 days on and 2 days off or 3 days on and 4 days off or 6 days on and 1 day off, or 6 days on and 1 day off. The 4 days on and 3 days off regimen or a modified regimen may be repeated for a 21-day cycle or a longer cycle. For example, Compound (1) or its pharmaceutically acceptable salt may be administered consecutively on each of the following four days during a 21-day period: days 1 through 4, days 8 through 11, and days 15 through 18, with a prescribed rest on days 5 through 7, days 12 through 14, and days 19 through 21. Such intermittent administration is applicable also to the combination therapies where Compound (1) or its pharmaceutical acceptable salt is administered in combination with one or more agents.

The lifetime cancer risk in germline PTEN mutations is also high in pediatric patients as well as adults. The prospective age-adjusted cancer incidence study recruited 368 patients with germline PTEN mutations and 27% of them (98 patients) were pediatric patients (<18 years of age). The study indicates that the cancer risks for melanoma and thyroid cancer are high in pediatric patients with germline PTEN mutations and recommends annual thyroid ultrasound and skin examination from diagnosis as cancer surveillance. See Tan, Clin Cancer Res. 2012; 18(2):400-407. In pediatric and adolescent subjects, it may be desirable to adjust both the dosing and dosing schedule as desired to account for lower body mass as compared to adults. The dose adjustment can be achieved by titrating the dose beginning at a low dose and then conducting dose escalation until adverse events preclude higher dosing and may require lowering the dose or until the target dose is achieved.

Before commencing treatment determination is made as to whether the subject has either one or more germline or somatic mutations of the PTEN gene. Germline mutations may be determined from family history of cancers involving mutation of the PTEN gene, by genotyping the subject or analyzing any tissue sample from the patient including blood or tumor sample taken from the subject for PTEN mutations where the subject has already been determined to possess or likely to possess a germline mutation of the PTEN gene. Somatic mutations are determined by analyzing a tumor sample for one or more PTEN inactivating mutations. If the subject has either a germline mutation or a somatic mutation, treatment with Compound (1) or its pharmaceutically acceptable salt is appropriate.

The “treatment” of malignant tumors in the present application includes post-operative auxiliary chemotherapy that is performed to prevent recurrence of tumors after surgically removing tumors, and pre-operative auxiliary chemotherapy prior to surgery to surgically remove tumors. The treatment includes treatment for the purpose of healing or remission of a disease, or for the purpose of suppressing or alleviating the progression of the disease or the relaxation of the symptoms. The treatment includes administration of a drug before or after a surgical procedure, or administration of a drug to be performed during or after radiation therapy or as an adjuvant therapy to prevent recurrence of the tumor in a patient where other treatments such as surgery have rendered the patient cancer-free.

An embodiment of the present disclosure provides an anti-cancerous tumor agent comprising Compound (1) or its pharmaceutically acceptable salt for treating a subject with a cancerous tumor which exhibits an inactivating PTEN mutation.

An embodiment of the present disclosure provides a pharmaceutical comprising Compound (1) or its pharmaceutically acceptable salt for treating a subject with a cancerous tumor which exhibits an inactivating PTEN mutation.

An embodiment of the present disclosure provides Compound (1) or its pharmaceutically acceptable salt for use in treating a subject with a cancerous tumor which exhibits an inactivating PTEN mutation.

An embodiment of the present disclosure provides use of Compound (1) or its pharmaceutically acceptable salt for treating a subject with a cancerous tumor which exhibits an inactivating PTEN mutation.

An embodiment of the present disclosure provides use of Compound (1) or its pharmaceutically acceptable salt for producing an anti-cancerous tumor agent used administering to a subject with a cancerous tumor which exhibits an inactivating PTEN mutation.

An embodiment of the present disclosure provides Compound (1) or its pharmaceutically acceptable salt for use in treating a subject with a cancerous tumor which exhibits an inactivating PTEN mutation; and administering Compound (1) or its pharmaceutically acceptable salt daily for at least 21 days to the subject. Preferably, the administering includes a dosing of Compound (1) or its pharmaceutically acceptable salt at 8, 12, 16, or 20 mg/day as the free base and the cancerous tumor includes head and neck cancer, breast cancer, renal cell carcinoma, genital cancer, gastrointestinal cancer, skin cancer, a brain tumor in the embodiment. More preferably, the administering includes a dosing of Compound (1) or its pharmaceutically acceptable salt at 16 or 20 mg/day once daily as the free base and the cancerous tumor includes more preferably breast cancer, thyroid cancer, renal cell carcinoma, endometrial cancer, colorectal cancer, melanoma, and glioblastoma in the embodiment. Even more preferably, the subject has a PTEN gene with a germline inactivating mutation in the embodiment.

An embodiment of the present disclosure provides Compound (1) or its pharmaceutically acceptable salt for use in treating a subject with a cancerous tumor which exhibits an inactivating PTEN mutation; and administering Compound (1) or its pharmaceutically acceptable salt 4 consecutive days followed by a 3-day drug holiday in a week for at least 21 days to the subjects in the embodiment. Preferably, the administering includes a dosing of Compound (1) or its pharmaceutically acceptable salt at 16, 20, 24, or 28 mg/day as the free base and the cancerous tumor includes head and neck cancer, breast cancer, renal cell carcinoma, genital cancer, gastrointestinal cancer, skin cancer, a brain tumor in the embodiment. More preferably, the administering includes a dosing of Compound (1) or its pharmaceutically acceptable salt at 24 mg/day as the free base and the cancerous tumor includes more preferably breast cancer, thyroid cancer, renal cell carcinoma, endometrial cancer, colorectal cancer, melanoma, and glioblastoma in the embodiment. Even more preferably, the subject has a PTEN gene with a germline inactivating mutation in the embodiment.

An embodiment of the present disclosure provides Compound (1) or its pharmaceutically acceptable salt for use in treating a subject with a cancerous tumor which exhibits a germline PTEN mutation; and administering Compound (1) or its pharmaceutically acceptable salt to the subject. Preferably, the cancerous tumor includes more preferably breast cancer, thyroid cancer, renal cell carcinoma, endometrial cancer, colorectal cancer, melanoma, and glioblastoma in the embodiment. More preferably, further comprising administering Compound (1) or its pharmaceutically acceptable salt daily or 4 consecutive days followed by a 3-day drug holiday in a week for at least 21 days in the embodiment. Even more preferably, the administering Compound (1) or its pharmaceutically acceptable salt daily at 16 or 20 mg/day in the embodiment. Another even more preferably, the administering Compound (1) or its pharmaceutically acceptable salt 4 days in a week at 24 mg/day in the embodiment.

An embodiment of the present disclosure provides Compound (1) or its pharmaceutically acceptable salt for use in treating a subject with a breast cancer which exhibits a germline PTEN mutation; and administering Compound (1) or its pharmaceutically acceptable salt to the subject. Preferably, further comprising administering Compound (1) or its pharmaceutically acceptable salt daily or 4 consecutive days followed by a 3-day drug holiday in a week for at least 21 days in this embodiment. More preferably, administering Compound (1) or its pharmaceutically acceptable salt daily for at least 21 days at 16 or 20 mg/day as the free base in the embodiment. Another more preferably, administering Compound (1) or its pharmaceutically acceptable salt 4 consecutive days followed by a 3-day drug holiday in a week for at least 21 days at 24 mg/day as the free base in this embodiment.

An embodiment of the present disclosure provides Compound (1) or its pharmaceutically acceptable salt for use in treating a subject with a thyroid cancer which exhibits a germline PTEN mutation; and administering Compound (1) or its pharmaceutically acceptable salt to the subject. Preferably, further comprising administering Compound (1) or its pharmaceutically acceptable salt daily or 4 consecutive days followed by a 3-day drug holiday in a week for at least 21 days in this embodiment. More preferably, administering Compound (1) or its pharmaceutically acceptable salt daily for at least 21 days at 16 or 20 mg/day as the free base in the embodiment. Another more preferably, administering Compound (1) or its pharmaceutically acceptable salt 4 consecutive days followed by a 3-day drug holiday in a week for at least 21 days at 24 mg/day in this embodiment. Even more preferably, administering Compound (1) or its pharmaceutically acceptable salt once daily for at least 21 days at 20 mg/day as the free base in the embodiment.

An embodiment of the present disclosure provides Compound (1) or its pharmaceutically acceptable salt for use in treating a subject with a renal cell carcinoma which exhibits a germline PTEN mutation; and administering Compound (1) or its pharmaceutically acceptable salt to the subject. Preferably, further comprising administering Compound (1) or its pharmaceutically acceptable salt daily or 4 consecutive days followed by a 3-day drug holiday in a week for at least 21 days in this embodiment. More preferably, administering Compound (1) or its pharmaceutically acceptable salt daily for at least 21 days at 16 or 20 mg/day as the free base in the embodiment. Another more preferably, administering Compound (1) or its pharmaceutically acceptable salt 4 consecutive days followed by a 3-day drug holiday in a week for at least 21 days at 24 mg/day in this embodiment.

An embodiment of the present disclosure provides Compound (1) or its pharmaceutically acceptable salt for use in treating a subject with endometrial cancer which exhibits a germline PTEN mutation; and administering Compound (1) or its pharmaceutically acceptable salt to the subject. Preferably, further comprising administering Compound (1) or its pharmaceutically acceptable salt daily or 4 consecutive days followed by a 3-day drug holiday in a week for at least 21 days in this embodiment. More preferably, administering Compound (1) or its pharmaceutically acceptable salt daily for at least 21 days at 16 or 20 mg/day as the free base in the embodiment. Another more preferably, administering Compound (1) or its pharmaceutically acceptable salt 4 consecutive days followed by a 3-day drug holiday in a week for at least 21 days at 24 mg/day in this embodiment.

An embodiment of the present disclosure provides Compound (1) or its pharmaceutically acceptable salt for use in treating a subject with colorectal cancer which exhibits a germline PTEN mutation; and administering Compound (1) or its pharmaceutically acceptable salt to the subject. Preferably, further comprising administering Compound (1) or its pharmaceutically acceptable salt daily or 4 consecutive days followed by a 3-day drug holiday in a week for at least 21 days in this embodiment. More preferably, administering Compound (1) or its pharmaceutically acceptable salt daily for at least 21 days at 16 or 20 mg/day as the free base in the embodiment. Another more preferably, administering Compound (1) or its pharmaceutically acceptable salt 4 consecutive days followed by a 3-day drug holiday in a week for at least 21 days at 24 mg/day in this embodiment.

An embodiment of the present disclosure provides Compound (1) or its pharmaceutically acceptable salt for use in treating a subject with melanoma which exhibits a germline PTEN mutation; and administering Compound (1) or its pharmaceutically acceptable salt to the subject. Preferably, further comprising administering Compound (1) or its pharmaceutically acceptable salt daily or 4 consecutive days followed by a 3-day drug holiday in a week for at least 21 days in this embodiment. More preferably, administering Compound (1) or its pharmaceutically acceptable salt daily for at least 21 days at 16 or 20 mg/day as the free base in the embodiment. Another more preferably, administering Compound (1) or its pharmaceutically acceptable salt 4 consecutive days followed by a 3-day drug holiday in a week for at least 21 days at 24 mg/day in this embodiment.

An embodiment of the present disclosure provides Compound (1) or its pharmaceutically acceptable salt for use in treating a subject with melanoma which exhibits a germline PTEN mutation; and administering Compound (1) or its pharmaceutically acceptable salt to the subject. Preferably, further comprising administering Compound (1) or its pharmaceutically acceptable salt daily or 4 consecutive days followed by a 3-day drug holiday in a week for at least 21 days in this embodiment. More preferably, administering Compound (1) or its pharmaceutically acceptable salt daily for at least 21 days at 16 or 20 mg/day as the free base in the embodiment. Another more preferably, administering Compound (1) or its pharmaceutically acceptable salt 4 consecutive days followed by a 3-day drug holiday in a week for at least 21 days at 24 mg/day in this embodiment.

An embodiment of the present disclosure provides Compound (1) or its pharmaceutically acceptable salt for use in treating a subject with a breast cancer which exhibits harboring PIK3CA mutation; and administering Compound (1) or its pharmaceutically acceptable salt to the subject. Preferably, further comprising administering Compound (1) or its pharmaceutically acceptable salt daily for at least 21 days at 16 or 20 mg/day as the free base in the embodiment. More preferably, administering Compound (1) or its pharmaceutically acceptable salt daily for at least 21 days at 20 mg/day as the free base in the embodiment.

Compound (1) or its pharmaceutically acceptable salt may be used in various dosage forms according to the purpose of treatment. The form can be exemplified by an oral agent (tablet, coated tablet, powder, granule, capsule, liquid agent, etc.), an injection agent, a suppository, a patch, an ointment, and the like. In the case of Compound (1) or its pharmaceutically acceptable salt, an oral formulation is preferable. These formulations can be formulated using a pharmaceutically acceptable carrier or the like by desirable formulation methods.

Examples of pharmaceutically acceptable carriers include various general-purpose agents such as excipients, binders, disintegrating agents, lubricants, diluents, dissolution aids, suspending agents, isotonic agents, pH adjusters, buffers, stabilizers, colorants, flavoring agents, flavoring agents, and the like.

Examples of excipients include lactose, sucrose, D-mannitol, glucose, starch, calcium carbonate, kaolin, microcrystalline cellulose, and silicic acid anhydride.

Examples of binders include water, ethanol, 1-propanol, 2-propanol, simple syrup, liquid glucose, liquid a-starch, liquid gelatin, D-mannitol, carboxymethyl cellulose, hydroxypropyl cellulose, hydroxypropyl starch, methyl cellulose, ethyl cellulose, shellac, calcium phosphate, and polyvinylpyrrolidone.

Examples of disintegrants include dry starch, sodium alginate, agar powder, sodium hydrogen carbonate, calcium carbonate, sodium lauryl sulfate, stearic acid monoglyceride, and lactose.

Examples of lubricants include purified talc, sodium stearate, magnesium stearate, borax, and polyethylene glycol.

Examples of colorants include titanium oxide, and iron oxide.

Examples of sweetening/flavoring agents include sucrose, wild orange peel, citric acid, tartaric acid, and the like.

If desired, an enteric coating or a coating to increase the persistence of effects can be provided by methods desirable for oral preparations. Examples of such coating agents include hydroxypropyl methylcellulose, ethyl cellulose, hydroxymethyl cellulose, hydroxypropyl cellulose, polyoxy ethylene glycol, and Tween 80 (registered trademark).

Compound (1) or its pharmaceutically acceptable salt can be combined with one or more other antitumor agents such as those described in U.S. Pat. No. 9,375,434, the disclosure of which is incorporated herein by reference. These include antitumor antibiotics such as doxorubicin or epirubicin; alkylating agents such as cyclophosphamide or nimustine; platinum-based agents such as cisplatin, carboplatin, oxaliplatin; pyrimidine-based antimetabolites such as 5-fluorouracil (5-FU), tipiracil/trifluridine (Lonsurf) tegafur/gimeracil/oteracil potassium (i.e., a combination drug of tegafur, gimeracil, and oteracil potassium) (TS-1, general name “a combination drug of tegafur, gimeracil, and oteracil potassium” (product name: “Ti-Esu Wan”)), tegafur/uracil (a combination drug of tegafur and uracil) (UFT, general name “a combination drug of tegafur and uracil” (product name: “Yu-Efu-Ti”)), capecitabine, doxifluridine, 5-fluoro-2′-deoxy uridine (FdUrd), gemcitabine, or cytarabine; purine acid-based antimetabolites such as fludarabine, cladribine, or nelarabine; folic acid antimetabolites such as pemetrexed or methotrexate; plant alkaloid antitumor agents such as paclitaxel (product names: “taxol,” “abraxane,” etc.), docetaxel, irinotecan, or vincristine; low molecular-weight molecular target drugs such as gefitinib, erlotinib, lapatinib, everolimus, temsirolimus, selumetinib, trametinib, sorafenib, afatinib, regorafenib, dabrafenib, vemurafenib, bortezomib, or carfilzomib; antibody molecular target drugs such as trastuzumab (herceptin), cetuximab, bevacizumab, panitumumab, veltuzumab, or rituximab; metformin, dexamethasone, thalidomide, and lenalidomide. In particular the other antitumor agent may be one or more of paclitaxel, carboplatin, lapatinib, irinotecan, doxorubicin, everolimus, bortezomib, erlotinib, trastuzumab (herceptin), metformin, docetaxel, a combination drug of tegafur, gimeracil, and oteracil potassium, trastuzumab, rapamycin analogues, Lonsurf and futibatinib.

As used in the present application, the term “combination,” “combined,” “in combination with” or a variation thereof is intended to define a therapy involving the use of two or more compound/drug combinations. The term can refer to compounds/drugs that are administered as part of the same overall dosage schedule. The respective dosages of two or more compounds/drugs can be different. The compounds/drugs can be administered simultaneously or at different times. The compounds/drugs can be administered consecutively (e.g., before or after) or simultaneously in either the same pharmaceutical formulation (i.e., together) or in different pharmaceutical formulations (i.e., separately). Simultaneously in the same pharmaceutical formulation is as a single formulation, while simultaneously in different pharmaceutical formulations is as separate formulations.

Compound (1) can exist in a crystal form having an x-ray diffraction pattern of least 5 diffraction angles)(20±0.2° selected from 7.7°, 9.5°, 10.3°, 12.3°, 14.5°, 15.6°, 16.3°, 17.8°, 18.3°, 19.3°, 20.9°, 22.8°, 24.2°, 25.7°, 26.8°, 27.7°, 29.0°, and 30.1° in a powder x-ray diffraction spectrum. Preferably the crystal form has peaks at diffraction angles)(20±0.2° 7.7°, 9.5°, 10.3°, 12.3°, 14.5°, 15.6°, 16.3°, 17.8°, 18.3°, 20.9°, 22.8°, 24.2°, 25.7°, 26.8°, 27.7°, 29.0°, and 30.1°. A crystal meeting either of these criteria shows good stability when stored at 40° C. for six months which is indicative of a 24 month shelf life at room temperature. The method preparing this crystal form is described in U.S. Pat. No. 10,323,044 the content of which is incorporated herein in its entirety.

### EXAMPLE

A patient with a mutation of the PTEN is administered Compound (1) by the oral administration once daily of 16 mg of Compound (1). If the dose is well tolerated, it may be escalated in 4 mg increments up to a daily dose of 24 mg. The dosing is continued daily. Alternatively, the patient may receive orally from 24 mg to 32 mg/day in 4 mg increments for 4 days followed by a 3 day drug holiday in a week which may be repeated for 21 days for a cycle. Objective response was observed in one patient with endometrial cancer harboring PIK3CA mutation and 3 patients with ovarian clear cell carcinoma. The 21 day drug cycle may be repeated as desired.

The following examples report the results of phase 2 clinical studies where Compound 1 in tablet form was administered orally to patients with different tumor type. In this trial Compound 1 was administered once/day at a dosage of 16 mg/day or 20 mg/day for a 21 day cycle which was repeated as desired. The compound was also administered intermittently on a schedule of 4 days and 3 days off for 21 days per cycle. The cycle was repeated as desired identifies the adverse events in the trials.

The safety profile of compound 1 (up to 20 mg QD and 24 mg Intermittent dosing) in patients with advanced cancer was generally acceptable and clinically manageable. Of the 15 patients treated, the most notable treatment-related AE was rash and maculopapular rash, observed for 62.5% of patients; other frequently reported treatment-related AEs include fatigue (37.5%), hyperglycemia (31.3%), pruritus (25.0%), mucositis oral (18.8%), decreased appetite (18.8%), diarrhea (18.8%), and nausea (12.5%). A total of 8 patients (50%) experienced at least 1 treatment-related event of Grade ≥3; the most frequently reported event of this kind was rash and maculopapular rash (25.0%). Other commonly reported treatment-related Grade ≥3 AEs were hyperglycemia (12.5%), mucositis oral (12.5%), and neutrophil count decreased (12.5%).

Abbreviations
Cycle=21 days

Of the 15 patients treated, 4 had tumor shrinkage, PIDs 100-006, 200-005, 100-009 and 200-007, with patient 100-006 showing a complete response in the target lesion (Lymph Node). The reported shrinkages are over 50% for patient 100-006, about 10% for patients 100-009 and about 5% for patient 200-007.

The 21-day cycles will be repeated until the patient either has disease progression or experience unacceptable toxicities.

Obviously, numerous modifications and variations of the present invention are possible in light of the above teachings. It is therefore to be understood that within the scope of the appended claims, the invention may be practiced otherwise than as specifically described herein.

1. A method of treating a subject with a cancerous tumor, comprising:
determining if the cancerous tumor exhibits an inactivating PTEN mutation; and
administering to the subject with the cancerous tumor exhibiting the inactivating PTEN mutation trans-3-amino-1-methyl-3-(4-(3-phenyl-5H-imidazo[1,2-c]pyrido[3,4-e][1,3]oxazin-2-yl)phenylcyclobutanol or a pharmaceutically acceptable salt thereof.

2. The method of claim 1 wherein the inactivating PTEN mutation is somatic.

3. The method of claim 1 wherein the administering is daily for at least 21 days.

4. The method of claim 1 wherein the administering is for 4 consecutive days followed by a 3-day drug holiday in a week.

5. The method of claim 4 wherein the administering is repeated for a total period of at least 21 days.

6. A method of treating a subject with a cancerous tumor, comprising:
determining if the subject has a PTEN gene with a germline inactivating mutation; and
administering to the subject having the PTEN gene with the germline inactivating mutation trans-3-amino-1-methyl-3-(4-(3-phenyl-5H-imidazo[1,2-c]pyrido[3,4-e][1,3]oxazin-2-yl)phenylcyclobutanol or a pharmaceutically acceptable salt thereof.

7. The method of claim 6 wherein the administering is daily for at least 21 days.

8. The method of claim 6 wherein the administering is for 4 consecutive days followed by a 3-day drug holiday.

9. The method of claim 8 wherein the administering for 4 consecutive days followed by the 3-day drug holiday is repeated for a total period of at least 21 days.

10. An anti-cancerous tumor agent comprising trans-3-amino-1-methyl-3-(4-(3-phenyl-5H-imidazo[1,2-c]pyrido[3,4-e][1,3]oxazin-2-yl)phenylcyclobutanol or a pharmaceutically acceptable salt thereof for treating a subject with a cancerous tumor which exhibits an inactivating PTEN mutation.

11. Trans-3-amino-1-methyl-3-(4-(3-phenyl-5H-imidazo[1,2-c]pyrido[3,4-e][1,3]oxazin-2-yl)phenylcyclobutanol or a pharmaceutically acceptable salt thereof for use in treating a subject with a cancerous tumor which exhibits an inactivating PTEN mutation.

12. The method of claim 1 wherein the dose of trans-3-amino-1-methyl-3-(4-(3-phenyl-5H-imidazo[1,2-c]pyrido[3,4-e][1,3]oxazin-2-yl)phenylcyclobutanol or a pharmaceutically acceptable salt administered is a therapeutically effective amount.

13. The method of claim 12 wherein the amount trans-3-amino-1-methyl-3-(4-(3-phenyl-5H-imidazo[1,2-c]pyrido[3,4-e][1,3]oxazin-2-yl)phenylcyclobutanol or a pharmaceutically acceptable salt administered is from about 1 to 160 mg/day as the free base.
